(19) United States
(12) Patent Application Publication
(10) Pub. No.: US 2020/0009143 A1
(43) Pub. Date: Jan. 9, 2020

(54) REPURPOSING OF CANCER DRUGS FOR TREATMENT OF MYCOBACTERIUM

(71) Applicant: STC.UNM, Albuquerque, NM (US)

(72) Inventors: Graham Timmins, Albuquerque, NM (US); Ke Jian Jim Liu, Albuquerque, NM (US); Xixi Zhou, Albuquerque, NM (US)

(21) Appl. No.: 16/310,615

(22) PCT Filed: Jul. 24, 2017
(86) PCT No.: PCT/US2017/043481

§ 371 Date: Dec. 17, 2018

Related U.S. Application Data
(60) Provisional application No. 62/366,292, filed on Jul. 25, 2016.

(51) Int. Cl. A61K 31/5025

(52) U.S. Cl. CPC A61K 31/5025; A61K 45/06

(57) ABSTRACT

The present invention is directed to the discovery that pyrazinamide, a potent anti-tuberculosis agent acts through an entirely unexpected mechanism-through inhibition of the host enzyme poly ADP ribose polymerase (“PARP”). Thus, the present invention is directed to methods of treating: mycobacterial infections (Mycobacterium), especially M. tuberculosis using a PARP inhibitor, optionally in combination with at least one additional agent useful in the treatment of a mycobacterial infection, especially tuberculosis. Pharmaceutical compositions, especially including a pharmaceutical composition in oral or inhalation dosage form, comprising a inhibitor, optionally in combination with an additional anti-mycobacterial agent, especially an additional anti-tuberculosis agent represent additional embodiments of the present invention.

### RELATED APPLICATIONS

This application claims the benefit of priority of U.S. Provisional application Ser. No. 62/366,292, filed Jul. 25, 2016 of identical title, the entire contents of which application are incorporated by reference herein.

### FIELD OF THE INVENTION

The present invention is directed to the discovery that pyrazinamide, a potent anti-tuberculosis agent acts through an entirely unexpected mechanism-through inhibition of the host enzyme poly ADP ribose polymerase (“PARP”). Thus, the present invention is directed to methods of treating mycobacterial infections (Mycobacterium), especially M. tuberculosis using a PARP inhibitor, optionally in combination with at least one additional agent useful in the treatment of a mycobacterial infection, especially tuberculosis. Pharmaceutical compositions, especially including a pharmaceutical composition in oral or inhalation dosage form, comprising a PARP inhibitor, optionally in combination with an additional anti-mycobacterial agent, especially an additional anti-tuberculosis agent such as isoniazid, ethionamide, aminosalicyclic acid/aminosalicylate sodium, capreomycin sulfate, clofazimine, cycloserine, ethambutol hydrochloride, kanamycin sulfate, pyrazinamide, pyrazinoic acid, rifabutin, rifampin, rifapentine, streptomycin sulfate, gatifloxacin among others and pharmaceutically acceptable salts, alternative salts and mixtures thereof.

Thus, the principal invention of the present application is the use of PARP inhibitors (preferably, low toxicity PARP inhibitors such as veliparib, among others) in the treatment of mycobacterial infections, including tuberculosis, especially pyrazinamide resistant disease. Other embodiments of the present invention are directed to novel pharmaceutical compositions comprising at least one PARP inhibitor, optionally in combination with at least one additional active agent, especially including an anti-tuberculosis agent, often in oral or especially inhalation dosage form, which are useful to treat TB. The present invention is also directed to PARP inhibition screens to discover new TB drugs, and the measurement of host PARP activity to determine optimal therapy for TB.

### BACKGROUND AND OVERVIEW OF THE INVENTION

Pyrazinamide's anti-TB activity was first reported in 1952,^{18,19} and it remains a vitally-important TB drug due to its potent sterilizing activity.^{1,7} However, understanding the mechanisms of action of PZA has greatly lagged its discovery, and remains incomplete. The most longstanding and agreed upon explanation is it tracellular acidification and proton gradient uncoupling by POA diffusion across the cell wall,^{36,23} supported by the pH sensitive nature of PZA activity,²² although recent studies have come to alternate conclusions.²² Recent studies have shown that POA also inhibits bacterial ribosomal trans-translation,⁸ and aspartate decarboxylase and thus pantothenate production.⁹ However, recent suggestions that therapies directed at host targets should be sought for TM,²³ and previous attempts to delineate a host-directed mechanism for PZA,²⁴ led us to discover that POA, but not its parent prodrug PZA, is a potent inhibitor of PARP at levels that are readily achieved during conventional PZA therapy. Furthermore, there is a long-known antagonism between PZA and the TB drug isoniazid (NH) that only occurs in the host,¹²⁻¹⁴ but is not understood. Since PARP is the first host target of PZA elucidated, the inventors hypothesize the antagonism of POA-induced. PARP inhibition by INH (or metabolites) could be compounded by PARP activation by DNA damage from known INH derived reactive oxygen and reactive nitrogen species (ROS and RNS),²⁵ only in the host, to explain this antagonism.

PARP-1 is the first identified member of the PARP family of 18 proteins in humans that catalyze the polymerization of ADP-ribose units from donor NAD⁺ molecules onto target proteins. PARP has an N-terminal double zinc finger DNA binding domain, a nuclear localization signal, a central auto-modification domain and a C-terminal catalytic domain.²⁶ It plays multiple roles in cellular responses to genotoxic and oxidative insult. PARP-1 recognizes and binds to damaged DNA rapidly catalyzing the covalent attachment of poly-ADP-ribose (PAR) units to acceptor proteins such as histones and transcription related factors and on PARP-1 itself. This covalent modification of proteins is an immediate response to DNA damage, thus PARP-1 functions as a DNA damage sensor. DNA binding of PARP-1 has been estimated to increase enzymatic activity as much as 500-fold. This reaction uses NAD⁺ as a substrate and the associated decrease in cellular NAD⁺ and ATP levels contribute to cell death. The catalytic activity appears to mediate interaction of PARP-1 with other proteins and regulate the activity of proteins involved in chromatin structure and DNA metabolism including proteins involved in single strand break repair and base excision repair.

The present invention may be used to treat PZA resistance and prevent PZA-INH antagonism. Clinical outcomes for just PZA mono-resistant TB are significantly worse,²⁷ while PZA resistance is found in about 60% of all multidrug resistant (MDR) TB, strongly negatively affecting treatment outcomes in MDR-TB.²⁸ PZA resistance is overwhelmingly due to mutations in the genes for the activating pyrazinamidase, resulting in low levels of active POA. Since POA is also the active form that inhibits host PARP, pyrazinamidase mutations will also prevent PARP inhibition. However, there are a range of alternate PARP inhibitors that are being developed for oncology application that could be used, preferably those, such as veliparib (with recently finished phase III trials) that are not involved in trapping of PARP at DNA breakage sites,²⁹ and have much lower side effects both in animal models,³⁶ and clinical studies, while retaining high efficacy.³⁷ Similarly, using these structurally unrelated PARP inhibitors instead of PZA could prevent antagonism caused by INH, and maximize the effects of combination drug regimens in TB and MDR-TB.

The inventors were the first to ever demonstrate a relevant host target for PZA. Secondly, the inventors have amassed considerable experience in re-examining the actions of old TB drugs,^{15,17-32} and in using the knowledge to drive new approaches, including intellectual property.³³⁻³⁶ We have also pioneered the development of stable isotope breath tests of tuberculosis infections and drug sensitivity,^{32,37-40} and clinical trials have been successfully performed.⁴¹⁻⁴³ Overall, the approach to the present invention offers the opportunity to both resolve a host directed target of PZA activity, and to understand the long-known antagonism between PZA and INH that only occurs in the context of the host. Since clinically-available PARP inhibitors are developed, rapid translation of these compounds to both overcome PZA resistance and to prevent antagonism with INH in the treatment of mycobacterial infections, especially M. tuberculosis infections could be adapted for use in the present invention after appropriate preclinical work.

### BRIEF DESCRIPTION OF THE INVENTION

The inventors have discovered that pyrazinamide (PZA), one of the most important TB drugs, acts in an entirely novel way, through inhibiting the host enzyme PARP. PZA is a prodrug, activated to pyrazinoic acid (POA) by the TB enzyme pyrazinamidase, and mutations in this enzyme dominate important clinical PZA resistance, so mycobacterial formation of POA. The whole-cell of PARP inhibition is about 5 μM and 125 μM for POA and PZA respectively, showing that resistance through pyrazinamidase mutation will also lead to a failure of host PARP inhibition. This is the first elucidation of a host target for PZA, and points to the importance of its activity upon the host, and not just the Mycobacterium.

The primary invention is the use of PARP inhibitors (especially low toxicity PARP inhibitors such as veliparib) in the treatment of tuberculosis, especially pyrazinamide resistant disease. Other inventions include the use of inhaled PARP inhibitors to treat TB, PARP inhibition screens to discover new TB drugs, and the measurement of host PARP activity to determine optimal therapy for TB.

The present invention is directed to methods for the treatment of a Mycobacterium infection in a host, preferably a M. tuberculosis infection, especially a PZA resistant, multidrug resistant or recurrent infection in a human host, comprising-administering to a patient in need an effective amount of a PARP inhibitor (often a PARP1 and/or a PARP 2 inhibitor) as described herein, optionally in combination with an additional anti-tuberculosis agent. The present invention is also directed to pharmaceutical compositions which comprise an effective amount of at least one PARP inhibitor as described herein, optionally in combination with a PARP inhibitor as described herein. Pharmaceutical compositions are preferably formulated in inhalation dosage form with one or more PARP inhibitors alone or in combination with an additional anti-tuberculosis agent. In other pharmaceutical compositions, especially including oral dosage forms, the composition comprises an effective amount of at least one PARP inhibitor in combination with at least one anti-tuberculosis agent in an effective amount.

The present invention is also directed to PARP inhibition screens to discover new anti-tuberculosis drugs, and the measurement of host PARP activity to determine optimal therapy (active agents and concentrations of those agents including route of administration) for mycobacterial, including tuberculosis treatment, especially drag resistant and multiple drug resistant tuberculosis in one embodiment, the present invention is directed to an assay for determining the activity of a compound as a potential anti-tuberculosis agent comprising poly ADP ribose polymerase (PARP) in combination with a reporter which can measure the inhibition on PART of the unknown compound as evidenced by the reporter. In certain embodiments, the assay may be an ELISA assay. The reporter used in the PARP assay may be any reporter which is used in biological assays and is consistent with its use in combination with PARP and may include colorimetric, fluorescent, chemiluminescent and radioactive reporters, among others well known in the art. PARP inhibition assays which may be used in the present invention to determine whether a compound with unknown activity may be PARP inhibitory activity consistent with its use as a potential therapy or drug for treating a mycobacterial infection, especially including tuberculosis are known in the art and include DELFIA® PARP assays from Perkin Elmer, the PARP homogeneous inhibition assay kit from Trevigen, a PARP1 chemiluminescent assay kit (among several) from BPS Bioscience, Chemicon® PARP1 enzyme activity assay from Millipore Sigma. PARP in vivo Pharmacodynamic Assay from AMSBIO, a cell based TDP1 inhibitory assay as described by Murai, et al., DNA REPAIR, Volume 21, September 2014, Pages 177-182, a PARP Universal Colorimetric Assay from R&D systems, the PARP assays from Reaction Biology Corp., among others.

In another embodiment the present invention is directed to a method for determining the activity of a compound with unknown PARP activity as a potential anti-mycobacterial, especially an anti-tuberculosis agent comprising exposing PARP in the assay described above to a compound with unknown activity to be screened (“the test compound”), obtaining a response of the test compound with PARP as evidenced by a response of a reporter in the assay and comparing the response of the reporter with a predetermined measurement (standard) wherein a measurement of the test compound in the assay which is the same as, above or below the predetermined measurement is an indication that the compound is an inhibitor of PARP and a potential anti-mycobacterial/anti-tuberculosis agent. The predetermined measurement may be, for example, a response in the assay to a compound with known PARP activity (inhibitory or agonist activity, often inhibitory activity), such that a comparison may be made between the activity of the test compound and the predetermined measurement in assessing the potential value of the test compound as an anti-mycobacterial(anti-tuberculosis) agent.

### BRIEF DESCRIPTION OF THE FIGURES

FIG. 1 shows whole cell PARP inhibition by PZA and POA. PARP activity was activated in RAW 264.7 macrophage cells (by UV) that were pre-treated with PZA or POA for 1 hour and then PARP activity measured by ELISA as previously.² Replicate plates, n=3, mean±se. The whole-cell IC₅₀ for POA and PZA was 15 μM and 125 μM respectively.

FIG. 2 shows that a phagosomal source of POA in mycobacteria-infected macrohages causes high intracellular levels.

### DETAILED DESCRIPTION OF THE INVENTION

The following terms are used throughout the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “patient” or “subject” is used throughout the specification to describe an animal, preferably a human, to whom treatment, including prophylactic treatment, with the compositions according to the present invention is provided (a patient or subject in need). For treatment of those infections, conditions or disease states which are specific for a specific animal such as a human patient, the term patient refers to that specific animal. In many instances, diagnostic methods are applied to patients or subjects who are suspected of having cancer or an inflammatory disorder or who have cancer or an inflammatory disorder and the diagnostic method is used to assess the severity of the disease state or disorder.

The term “compound” is used herein to refer to any specific chemical compound disclosed herein and in particular, a PARP inhibitor or an additional anti-mycobacterial agent, especially an additional agent effective in the treatment of M. tuberculosis. Within its use in context, the term generally refers to a simile small molecule as disclosed herein, but in certain instances may also refer to other forms of the compound. The term compound includes active metabolites of compounds and/or pharmaceutically acceptable salts (including alternative pharmaceutically acceptable salts) thereof. Also included under the term “compound” are stereoisomers (e.g., diastereoisomers, enantiomers) and solvates (including hydrates) and polymorphs.

The term “effective amount” is used throughout the specification to describe concentrations or amounts of formulations or other components which are used in amounts, within the context of their use, to produce an intended effect according to the present invention, to inhibit PARP (e.g. PARP-1 and/or PARP-2, among others). The formulations or component(s) may be used to produce a favorable change in a disease or condition treated, whether that change is a remission of the effects of a disease state or condition, a favorable physiological result, a reversal or attenuation of a disease state or condition treated, the prevention or the reduction in the likelihood of a condition or disease-state occurring, depending upon the disease or condition treated. Where formulations are used in combination, each of the formulations is used in an effective amount, wherein an effective amount may include a synergistic amount. The amount of formulation used in the present invention may vary according to the nature of the formulation, the age and weight of the patient and numerous other factors which may influence the bioavailability and pharmacokinetics of the formulation, the amount of formulation which is administered to a patient generally ranges from about 0.001 mg/kg to about 50 mg/kg or more, about 0.5 mg/kg to about 25 mg/kg, about 0.1 to about 15 mg/kg, about 1 mg to about 10 mg/kg per day and as otherwise described herein. The person of ordinary skill may easily recognize variations in dosage schedules or amounts to be made during the course of therapy.

The term “prophylactic” is used to describe the use formulation described herein which reduces the likelihood of an occurrence of a condition or disease state in a patient or subject. The term “reducing the likelihood” refers to the fact that in a given population of patients, the present invention may be used to reduce the likelihood of an occurrence, recurrence or metastasis of disease in one or more patients within that population of all patients, rather than prevent, in all patients, the occurrence, recurrence or metastasis of a disease state.

The term “pharmaceutically acceptable” refers to a salt form or other derivative (such as an active metabolite or prodrug form) of the present compounds or a carrier, additive or excipient which is not unacceptably toxic to the subject to which it is administered.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, reduction in the likelihood or delay in the onset of the disease, etc. Treatment, as used herein, may encompass both therapeutic and prophylactic treatment, but is typically therapeutic, depending on the context of the treatment.

The term “coadministration” is used to describe the administration of two active compounds. Although the term coadministration preferably includes the administration of two active compounds to the patient at the same time, it is not necessary that the compounds actually be administered at the exact same time, only that amounts of compound will be administered to a patient or subject such that effective concentrations are found in the blood, serum or plasma, or in the pulmonary tissue at the same time. In the present invention, the term coadministration refers to the administration of a PARP inhibitor in combination with an anti-tuberculosis agent or the administration of a PARP inhibitor with a tuberculosis vaccine or during the period when the patient or subject is developing immunity to M. tuberculosis as a consequence of vaccine administration or immunogenic challenge.

The term “Mycobacterium infections” refers to infections caused by intracellular microorganisms of the genus Mycobacterium, including diseases caused by the species M. tuberculosis. M. africanum, M. bovis, M. bovis BCG, M. canetti, M. microti. M. caprae, M. pinnipedii, M. avium, and M. leprae. “Mycobacterium infections” include infections caused by members of the Mycobacterium tuberculosis complex, the Mycobacterium avium complex, the Mycobacterium gordonae elude, the Mycobacterium kansasii clade, the Mycobacterium nonchromogenicum/terrae clade, the mycolactone-producing mycobacteria, the Mycobacterium simiae clade, the Mycobacterium chelonae clade, the Mycobacterium fortuitum clade, the Mycobacterium parafortuitum clade and the Mycobacterium vaccae clade.

Mycobacterium infections include infections associated with nontuberculous mycobacteria (NTM), which are classified based on their growth rates. Rapidly growing NTM are categorized into pigmented and nonpigmented species. Mycobacterium fortuitum complex is nonpigmented and includes the M. fortuitum group and the Mycobacterium chelonae/abscessus group. The pigmented species are rarely associated in clinical disease and include Mycobacterium phlei, Mycobacterium aurum, Mycobacterium flavescens, Mycobacterium vaccae, Mycobacterium neoaurum, and Mycobacterium thermoresistible. Mycobacterium smegmatis may be either pigmented or nonpigmented.

“Mycobacterium infections” also include atypical mycobacterial infections. Mycobacterium avium complex (MAC) and Mycobacterium scrofulaceum are associated with lymphadenitis in immunocompetent children. MAC has also been associated with the pulmonary infection and bronchiectasis in elderly women without a preexisting lung disease. Pulmonary MAC infection in this population is believed to be due to voluntary cough suppression that results in stagnation of secretions, which is suitable for growth of the organisms. Mycobacterium ulcerans, the agent of a chronic ulcerative skin infection called Buruli ulcer, is widespread in Ghana, Cote d'Ivoire, Senegal, Uganda, and most central African countries.

The term “Tuberculosis” or “TB” is used to describe the infection caused by the infective agent “Mycobacterium tuberculosis” or “M. tuberculosis”, a tubercle bacillus bacteria. Tuberculosis is a potentially fatal contagious disease that can affect almost any part of the body but is most frequently an infection of the lungs. It is caused by a bacterial microorganism, the tubercle bacillus or Mycobacterium tuberculosis.

Tuberculosis is primarily an infection of the lungs, but any organ system is susceptible, so its manifestations may be varied. Effective therapy and methods of control and prevention of tuberculosis have been developed, but the disease remains a major cause of mortality and morbidity throughout the world. The treatment of tuberculosis has been complicated by the emergence of drug-resistant organisms, including multiple-drug-resistant tuberculosis, especially in those with HIV infection.

Mycobacterium tuberculosis, the causative agent of tuberculosis, is transmitted by airborne droplet nuclei produced when an individual with active disease coughs, speaks, or sneezes. When inhaled, the droplet nuclei reach the alveoli of the lung. In susceptible individuals the organisms may then multiply and spread through lymphatics to the lymph nodes, and through the bloodstream to other sites such as the lung apices, bone marrow, kidneys, and meninges.

The development of acquired immunity in 2 to 10 weeks results in a halt to bacterial multiplication. Lesions heal and the individual remains asymptomatic. Such an individual is said to have tuberculous infection without disease, and will show a positive tuberculin test. The risk of developing active disease with clinical symptoms and positive cultures for the tubercle bacillus diminishes with time and may never occur, but is a lifelong risk. Approximately 5% of individuals with tuberculous infection progress to active disease. Progression occurs mainly in the first 2 years after infection; household contacts and the newly infected are thus at risk.

Many of the symptoms of tuberculosis, whether pulmonary disease or extrapulmonary disease, are nonspecific. Fatigue or tiredness, weight loss, fever, and loss of appetite may be present for months. A fever of unknown n origin may be the sole indication of tuberculosis, or an individual may have an acute influenza-like illness. Erythema nodosum, a skin lesion, is occasionally associated with the disease.

The lung is the most common location for a focus of infection to flare into active disease with the acceleration of the growth of organisms. Infections in the lung are the primary focus of the present invention. There may be complaints of cough, which can produce sputum containing mucus, pus- and, rarely, blood. Listening to the lungs may disclose rates or crackles and signs of pleural effusion (the escape of fluid into the lungs) or consolidation if present. In many, especially those with small infiltration, the physical examination of the chest reveals no abnormalities.

Miliary tuberculosis is a variant that results from the blood-borne dissemination of a great number of organisms resulting in the simultaneous seeding of many organ systems. The meninges, liver, bone marrow, spleen, and genitourinary system are usually involved. The term miliary refers to the lung lesions being the size of millet seeds (about 0.08 in, or 2 mm). These lung lesions are present bilaterally. Symptoms are variable.

Extrapulmonary tuberculosis is much less common than pulmonary disease. However, in individuals with AIDS, extrapulmonary tuberculosis predominates, particularly with lymph node involvement, with some pulmonary impact. For example, fluid in the lungs and lung lesions are other common manifestations of tuberculosis in AIDS. The lung is the portal of entry, and an extrapulmonary focus, seeded at the time of infection, breaks down with disease occurring.

Development of renal tuberculosis can result in symptoms of burning on urination, and blood and white cells in the urine: or the individual may be asymptomatic. The symptoms of tuberculous meningitis are nonspecific, with acute or chronic fever, headache, irritability, and malaise.

A tuberculous pleural effusion can occur without obvious lung involvement. Fever and chest pain upon breathing are common symptoms. Bone and joint involvement results in pain and fever at the joint site. The most common complaint is a chronic arthritis usually localized to one joint. Osteomyelitis is also usually present. Pericardial inflammation with fluid accumulation or constriction of the heart chambers secondary to pericardial scarring are two other forms of extrapulmonary disease.

At present, the principal methods of diagnosis for pulmonary tuberculosis are the tuberculin skin test (an intracutancous injection of purified protein derivative tuberculin is performed, and the injection site examined for reactivity), sputum smear and culture, and the chest x-ray. Culture and biopsy are important in making the diagnosis in extrapulmonary disease.

The term “PARP inhibitor” refers to agents which inhibit poly ADP ribose polymerase or PARP, often PARP-1 and/or PARP-2. PARP inhibitors for use in the present invention include, for example, NU1025; 3-aminobenzamide; benzamide: picolinamide, 4-amino-1,8-naphthalimide; coumarin, 1,5-isoquinolinediol: 6(5H)-phenanthriddinone; 1,3,4,5,-tetrahydrobenzo(1,6)- and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-(3-(4-phenyl-3,6-dihydro-1 (2H)-pyridinyl)propyl)-4(3H)-quinazolinone: isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5 [4-(1-piperidinyl)(butoxy)-1(2H)-isoquinolone; CEP-6800; GB-15427; PJ34; DPQ: BS-201; BGB-290; BGP-15; BS401; CHP101: CHP102: E7016(EISAI), INH2BP; BS1201; BS1401; TIQ-A; NMS-P118; E7449; NVP-TNKS656; G007-LK; ME0328; AZD2461; UPF 1069; imidazobenzodiazepine PARP inhibitors (see Ferraris, et al., Bioorganic & Medicinal Chemistry, 11 (2003) 3695-3707), which is incorporated by reference herein; 8-hydroxy-2-methylquinazolinone (NU1025). CEP 9722, MK 4827 (Niraparib), LT-673; 3-aminobenzamide; ABT-888 (Veliparib); BSI-201 (Iniparib); Rucapavib (AG-014699); BMN-673 (Talazopirib); AZD2281 (Olaparib). INO-1001; A-966492; PJ-34; Niraparib (MK-4827), Arsenic inoxide (ATO) and the PARP1 inhibitors described in U.S. Pat. No. 8,445,537, which is incorporated by reference herein, pharmaceutically acceptable salts (including alternative salts) thereof and mixtures thereof. PARP inhibitors which show enhanced inhibitory activity of PARP with low toxicity, such as ABT-888 (Veliparib). BSI-201 (Iniparib). Rucaparib (AG-014699). BMN-673 (Talazoparib), AZD2281 (Olaparib), and Niraparib (MK-4827) may be preferred.

The term “additional anti-tuberculosis agent” refers to anti-mycobacterial agents such as isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin among others and pharmaceutically acceptable salts, alternative salts and mixtures thereof. In certain embodiments, pyrazinamide and pyrazinoic acid may also be included as additional anti-tuberculosis agents.

In one embodiment of our invention, a subject suffering from a Mycobacterium infection (e.g. a Mtb Infection, latent tuberculosis infection “LTBI” or MDR-TB) is administered a therapeutically effective amount of a PARP inhibitor, optionally in combination with one or more anti-mycobacterial agents such as pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin, among others and pharmaceutical salts/alternative pharmaceutical salts and mixtures thereof.

In another embodiment, the invention provides a pharmaceutical composition comprising a therapeutically effective amount of a PARP inhibitor and one or more additional anti-mycobacterial agents such as pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin among others and pharmaceutically acceptable salts and mixtures thereof. In one embodiment, the present invention comprises at least one PARP inhibitor in an effective amount for treating a tuberculosis infection in inhalation dosage form. In other embodiments, the PARP inhibitor(s) is combined with at least one anti-tuberculosis agent as otherwise described herein.

In still another embodiment, the invention provides a method of treating a subject who suffers from or who is at risk of developing a Mycobacterium infection (e.g. Mtb, MDR-TB, pyrazinamide-resistant TB or MDR-TB with pyrazinamide resistance), the method comprising administering to the subject a therapeutically effective amount of a PARP inhibitor, optionally in combination with at least one additional anti-mycobacterial agent as described above, wherein in some embodiments, the additional anti-mycobacterial agent is other than pyrazinamide or pyrazinoic acid.

In still another embodiment, the invention provides a method of treating a subject who suffers from a latent Mycobacterium infection (e.g., LTBI), the method comprising administering to the subject a therapeutically effective amount of a PARP inhibitor, optionally in combination with an additional anti-mycobacterial agent as described herein (in certain embodiments the additional anti-mycobacterial agent is other than pyrazinamide), wherein administration of the PARP inhibitor, optionally in combination with an additional anti-mycobacterial agent as described above, prevents the latent Mycobacterium infection from progressing to an active Mycobacterium infection.

In certain embodiments, a PARP inhibitor as described herein is co-administered with one or more antimycobacterial agents (e.g. anti-tuberculosis agents) selected from the group consisting of isoniazid, ethionamide, aminosalicyclic acid/aminosalicylate sodium, capreomycin sulfate, clofazimine, cycloserine, ethambutol hydrochloride (myambutol), kanamycin sulfate, pyrazinamide, pyrazinoic acid (in certain embodiments not used), rifabutin, rifampin, rifapentine, streptomycin sulfate, gatifloxacin and mixtures thereof, or pharmaceutically acceptable salts or alternative salts thereof.

Although the compositions described herein may be administered by any route of administration, including parenteral, topical or oral administration among others, in preferred aspects of the invention, the PARP inhibitor and optional additional anti-tuberculosis agent is preferably administered orally or alternatively, directly to the lungs of the subject via pulmonary administration, including intratracheal administration.

Formulations of the invention may include a pharmaceutically acceptable diluent, carrier, solubilizer, emulsifier, preservative and/or adjuvant. Acceptable formulation materials preferably are nontoxic to recipients at the dosages and concentrations employed. The pharmaceutical formulations may contain materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antioxidants (such as ascorbic acid, sodium sulfite or sodium hydrogen-sulfite); buffers (such as borate, bicarbonate, Tris-HCl, citrates, phosphates or other organic acids): bulking agents (such as mannitol or glycine); chelating agents (such as ethylenediamine tetraacetic acid (EDTA)); complexity agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); adoring, flavoring and diluting agents: emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, polyethylene glycol (PEG), sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol), delivery vehicles; diluents; excipients and/or pharmaceutical adjuvants. See, for example, REMINGTON'S PHARMACEUTICAL SCIENCES, 18.sup.th Edition. (A. R. Gennaro, ed.), 1990, Mack Publishing Company.

Optimal pharmaceutical formulations can be determined by one skilled in the art depending upon, for example, the intended route of administration, delivery format and desired dosage. See, for example, REMINGTON'S PHARMACEUTICAL SCIENCES, Id. Such formulations may influence the physical state, stability, rate of in vivo release and rate of in vivo clearance of the antibodies of the invention.

Primary vehicles or carriers in a pharmaceutical formulation can include, but are not limited to, water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical formulations can comprise Tris buffer of about pH 7.0-8.5, or acetate buffer of about pH 4.0-5.5, which may further include sorbitol or a suitable substitute. Pharmaceutical formulations of the invention may be prepared for storage by mixing the selected composition having the desired degree of purity with optional formulation agents (REMINGTON'S PHARMACEUTICAL SCIENCES, Id.) in the form of a lyophilized cake or an aqueous solution. Further, the formulations may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical formulations of the invention can be delivered parenterally. When parenteral administration is contemplated, the therapeutic formulations for use in this invention may be in the form of a pyrogen-free, parenterally acceptable aqueous solution. Preparation involves the formulation, which may provide controlled or sustained release of the product which may then be delivered via a depot injection. Formulation with hyaluronic acid has the effect of promoting sustained duration in the circulation.

Formulations of the invention can be delivered through the digestive tract, such as orally. The preparation of such pharmaceutically acceptable compositions is within the skill of the an. Formulations disclosed herein that ate administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption. Diluents, flavorings, low melting point waxes, vegetable oils, lubricants, suspending agents, tablet disintegrating agents, and binders may also be employed.

A formulation may involve an effective quantity of a microparticle containing formulation as disclosed herein in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin:, or acacia; or lubricating agents such as magnesium stearate, stearic acid, or talc.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the formulation of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

Administration routes for formulations of the invention include orally, through injection by intravenous, intraperitoneal, intracerebral (intra-parenchymal), intracerebroventricular, intramuscular, intra-ocular, intraarterial, intraportal, or intralesional routes: by sustained release systems or by implantation devices. The pharmaceutical formulations may be administered by bolus injection or continuously by infusion, or by implantation device. The pharmaceutical formulations also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

The pharmaceutical composition of the invention for pulmonary administration is often used as an inhalant. The composition can be formed into dry powder inhalants, inhalant suspensions, inhalant solutions, encapsulated inhalants and like known forms of inhalants. Such forms of inhalants can be prepared by filling the pharmaceutical composition of the invention into an appropriate inhaler such as a metered-dose inhaler, dry powder inhaler, atomizer bottle, nebulizer etc. before use. Of the above thrills of inhalants, powder inhalants may be preferable.

When the pharmaceutical composition of the invention is used in the form of a powder, the mean particle diameter of the powder is not especially limited but, in view of the residence of the particles in the lungs, is preferably that the particles fall within the range of about 0.1 to 20 μm, and particularly about 1 to 5 μm. Although the particle size distribution of the powder pharmaceutical composition of the invention is not particularly limited, it is preferable that particles having a size of about 25 μm or more account for not more than about 5% of the particles, and preferably, 1% or less to maximize delivery into the lungs of the subject.

The pharmaceutical composition in the form of a powder of the invention can be produced by, for example, using the drying-micronization method, the spray drying method and standard pharmaceutical methodology well known in the art.

By way of example without limitation, according to the drying-pulverization method, the pharmaceutical composition in the form of a powder can be prepared by drying an aqueous solution (or aqueous dispersion) containing the active(s) and excipients which provide for immediate release pulmonary tissue and microparticulating the dried product. Stated more specifically, after dissolving (or dispersing) a pharmaceutically acceptable carrier, additive or excipient in an aqueous medium, the active(s) in effective amount is added and dissolved (or dispersed) by stirring using a homogenizer, etc. to give an aqueous solution (or aqueous dispersion). The aqueous medium may be water alone or a mixture of water and a lower alcohol. Examples of usable lower alcohols include methanol, ethanol, 1-propanol, 2-propanol and like water-miscible alcohols. Ethanol is particularly preferable. After the obtained aqueous solution (or aqueous dispersion) is dried by blower, lyophilization, etc., the resulting product is pulverized or micropaniculated into fine particles using jet mills, ball mills or like devices to give a powder having the above mean particle diameter. If necessary, additives as mentioned above may be added in any of the above steps.

According to the spray-drying method, the pharmaceutical composition in the form of a powder of the invention can be prepared, for example, by spray-drying an aqueous solution (or aqueous dispersion) containing PARP inhibitor(s) and optional anti/tuberculosis agent(s), excipients, additives or carriers for microparticulation. The aqueous solution (or aqueous dispersion) can be prepared following the procedure of the above drying-micronization method. The spray-drying process can be performed using a known method, thereby giving a powdery pharmaceutical composition in the form of globular particles with the above-mentioned mean particle diameter.

The inhalant suspensions, inhalant solutions, encapsulated inhalants, etc. can also be prepared using the pharmaceutical composition in the form of a powder produced by the drying-micronization method, the spray-drying method and the like, or by using a carrier, additive or excipient and ethionamide/prothionamide that can be administered via the lungs, according to known preparation methods.

Furthermore, the inhalant comprising the pharmaceutical composition of the invention is preferably used as an aerosol. The aerosol can be prepared, for example, by filling the pharmaceutical composition of the invention and a propellant into an aerosol container. If necessary, dispersants, solvents and the like may be added. The aerosols may be prepared as 2-phase systems, 3-phase systems and diaphragm systems (double containers). The aerosol can be used in any form of a powder, suspension, solution or the like.

Examples of usable propellants include liquefied gas propellants, compressed gases and the like. Usable liquefied gas propellants include, for example, fluorinated hydrocarbons (e.g., CFC substitutes such as HCFC-22, HCFC-123, HFC-134a, HFC-227 and the like), liquefied petroleum, dimethyl ether and the like. Usable compressed gases include, for example, soluble gases (e.g., carbon dioxide, nitric oxide), insoluble gases (e.g., nitrogen) and the like.

The dispersant and solvent may be suitably selected from the additives mentioned above. The aerosol can be prepared, for example, by a known 2-step method comprising the step of preparing the composition of the invention and the step of filling and sealing the composition and propellant into the aerosol container.

As a preferred embodiment of the aerosol according to the invention, the following aerosol can be mentioned: Examples of the compounds to be used include PARP inhibitor(s) and optional anti-tuberculosis agent(s). As propellants, fluorinated hydrocarbons such as HFC-134a, HFC-227 and like CFC substitutes are preferable. Examples of usable solvents include water, ethanol, 2-propanol and the like. Water and ethanol are particularly preferable. In particular, a weight ratio of water to ethanol in the range of about 0:1 to 10:1 may be used.

The aerosol of the invention contains excipient in an amount ranging from about 0.01 to about 10⁴ wt. % (preferably about 0.1 to 10³ wt. %), propellant in an amount of about 10² to 10⁷ wt. % (preferably about 10³ to 10⁶ wt. %), solvent in an amount of about 0 to 10⁶ wt. % (preferably about 10 to 10⁴ wt %), and dispersant in an amount of 0 to 10³ wt. % (preferably about 0.01 to 10² wt. %), relative to the weight of active compound which is included in the final composition.

The pharmaceutical compositions of the invention are safe and effective for use in the treatment or prevention (reducing the likelihood) of a Mycobacterial infection, especially a M. tuberculous infection according to the present invention. Although the dosage of the composition of the invention may vary depending on the type of active substance administered, the route of administration, as well as the nature (size, weight, etc.) of the subject to be treated, the composition is administered in an amount effective for allowing the pharmacologically active substance to be effective. For example, the composition is preferably administered such that the active ingredient can be given to a human adult in a dose of about 0.001 to about 750 mg or more, about 0.01 mg to about 500 mg, about 0.05 mg to about 400 mg, about 0.1 mg to about 350 mg, about 0.5 mg to about 300 mg, about 1 to about 250 mg.

The amount of a PARP inhibitor that may be combined with the carrier materials to produce a single dosage form will vary depending upon the host treated and the particular mode of administration. Preferably, the compositions should be formulated so that a therapeutically effective dosage of between about 0.1 and 25 mg kg, about 0.5 to about 15 mg/kg of the patient day, preferably between 1 mg and 25 mg/kg or about 5 mg to about 15 mg/kg of the patient/day of the PARP inhibitor can be administered to a patient receiving these compositions. Preferably, pharmaceutical compositions in dosage form according to the present invention comprise a therapeuticially effective amount of at least about 5-10 mg of a PARP inhibitor, at least about 25 mg of PARP inhibitor, at least 50 mg of a PARP inhibitor, at least 60 mg of a PARP inhibitor, at least 75 mg of a PARP inhibitor, at least 100 mg of a PARP inhibitor, at least 150 mg of a PARP inhibitor, at least 200 mg of a PARP inhibitor, at least 250 mg of a PARP inhibitor, at least 300 mg of a PARP inhibitor, about 350 mg of a PARP inhibitor, about 400 mg of a PARP inhibitor, about 500 mg of a PARP inhibitor, about 750 mg of a PARP inhibitor, about 1 g (1,000 mg) of a PARP inhibitor, alone or in combination with a therapeutically effective amount of at least one additional anti-tuberculosis agent. Exemplary additional anti-tuberculosis agents which may be used in pharmaceutical compositions include one or more of isoniazid, ethionamide, aminosalicyclic acid/aminosalicylate sodium, capreomycin sulfate, clofazimine, cycloserine, ethambutol hydrochloride (myambutol), kanamycin sulfate, rifabutin, rifampin, rifapentine, streptomycin sulfate, gatifloxacin or pharmaceutically acceptable salts or alternative salts and mixtures thereof, all in therapeutically effective amounts. Pyrazinamide and/or pyrazinoic acid, although excluded in many embodiments, may also be used in certain embodiments according to the present invention where pyrazinamide and/or pyrazinoic acid resistance is not seen in the patient to be treated.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

The form of the pharmaceutical composition of the invention such as a powder, solution, suspension etc. may be suitably selected according to the type of substance to be administered.

As an administration route, direct inhalation via the mouth using an inhaler may be preferable. Since the pharmaceutical composition of the invention allows direct local administration into the airways and in particular, directly to pulmonary tissue, the active substance contained therein produces immediate effects. Furthermore, the composition is formulated as an immediate release product so that anti-tuberculosis activity can begin soon alter administration.

The invention is illustrated further in the following non-limiting examples.

### EXAMPLES

The following experiments are proposed:

Determining the Role of PARP inhibition by POA and PZA in the mycobacteria-macrophage interface. Using biochemical and cell biological approaches the inventors examine the consequences of PARP inhibition in enzyme and cell based systems.

Exploring antagonism of PARP inhibition by Isoniazid and its metabolites. Biochemical and cellular approaches explore potential antagonistic interactions of INH and metabolites upon PARP inhibition, and PARP activation by INH-derived reactive species.

Successful completion resolves longstanding questions upon PZA activity, antagonism by other TB drugs, and present PARP as a new host target in TB treatment.

### Pyrazinamide Activity Through PARP Inhibition

### Pyrazinamide Activities Remain Unresolved

Pyrazinamide (PZA) is one of the most important drugs against tuberculosis (TB) because of its potent sterilizing activity,¹ and is a mainstay of drug regimens.^{3,4} PZA is a prodrug activated by the enzyme pyrazinamidase (PZAse) to the active metabolite pyrazinoic acid (POA),^{5,6} and mutations in PZAse dominate clinical resistance. The most longstanding explanation of PZA activity is that POA in acidic milieu causes intracellular acidification and collapse of the proton motive force across the mycobacterial plasma membrane.⁷ More recently POA has been shown to inhibit additional bacterial targets: trans-translation,⁸ and mycobacterial aspartate decarboxylase.⁹ However, prompted by the recently elucidated role of host bioactivation of pyrazinamide,¹⁰ the inventors searched for relevant host targets whose activity could be modulated by POA, to question whether host-mediated effects may also be involved in PZA activity.

### POA Inhibits Poly ADP Ribose Polymerase (PARP) at Clinically Relevant Concentrations

Due to chemical similarity of NAD⁺ and PZA/POA (see chemical structures, below) the inventors focused upon NAD⁺-dependent host processes, including PARP, an important enzyme family that catalytically use NAD⁺ to poly-ADP-ribosylate a variety of targets. The inventors found that POA inhibits whole-cell PARP activity in relevant cell lines with an IC₅₀ of 15 μM, a value much lower than steady state plasma levels of POA, 300 μM, achieved in patients treated with PZA.¹⁰ Furthermore, not only is PARP inhibition by POA likely in patients, but PARP inhibition by another analogous agent (3-aminobenzamide, 3-AB) strongly inhibited mycobacteria-induced.

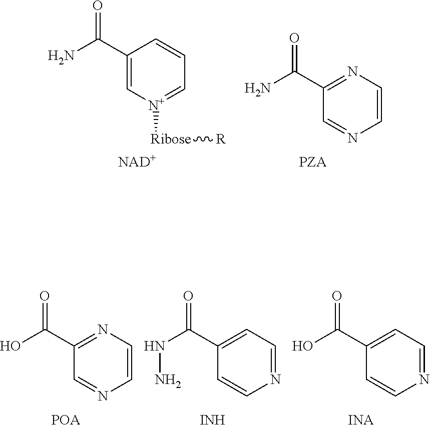

macrophage necrosis.¹¹ Therefore, PARP inhibition by POA could modulate the mycobacterial-macrophage interface that is centrally important in TB. Furthermore, since PARP is the lint host-target of PZA elucidated, the long-known antagonism between PZA and INH that occurs only in the host¹²⁻¹⁴ may be due to antagonism of PARP inhibition by POA by the structurally-related TB drug isoniazid (INH), its metabolites (e.g. isonicotinic acid INA) or PARP activation by INH-derived oxidants.^{15,17}

Pursuant to the present invention, the inventors hypothesize that POA inhibition of macrophage PARP contributes to the antimycobacterial activity of pyrazinamide by preventing NAD⁺ depletion and necrosis. Further, this is antagonized by isoniazid, its metabolites or isoniazid-derived oxidants.

Preliminary Studies. The inventors show that published steady state plasma levels of POA in patients treated with PZA exceed the IC₅₀ for PARP inhibition in whole-cell assays.

### PARP Inhibition by POA is Titratable and Achievable at Known Plasma Levels.

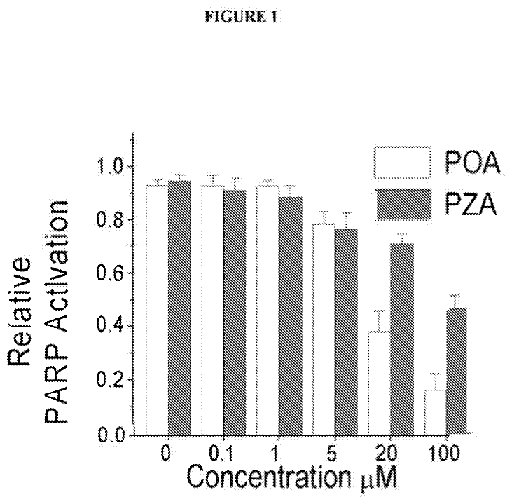

PZA is a prodrug activated to POA, the active form. Known steady state peak plasma levels of POA of 300 μM are known to be achieved in TB patients treated with PZA, while peak levels of PZA are about 500 μM,¹⁰ whilst both POA and PZA appear to achieve wide distribution in human granulomas.¹¹ The inventors examined inhibition of PARP (induced by UV) in a number of cell types by both POA and PZA, including macrophage cell lines (e.g. RAW 264.7 macrophages) that are most relevant to the host-pathogen interface in TB. FIG. 1 shows the dose response of PARP inhibition for both POA and PZA in RAW 264.7 macrophages, with IC₅₀ of about 15 and 125 μM respectively. Known peak plasma levels of POA and PZA of 300 μM and 500 μM in TB patients treated with PZA,¹⁰ mean the ratio of plasma Cₘₐₓ/IC₅₀ is 20 for POA while it is only 4 for PZA, so that inhibition of PARP by POA should be dominant when it is formed by PZase.

Macrophage POA Levels are Likely to be Even Higher than Plasma

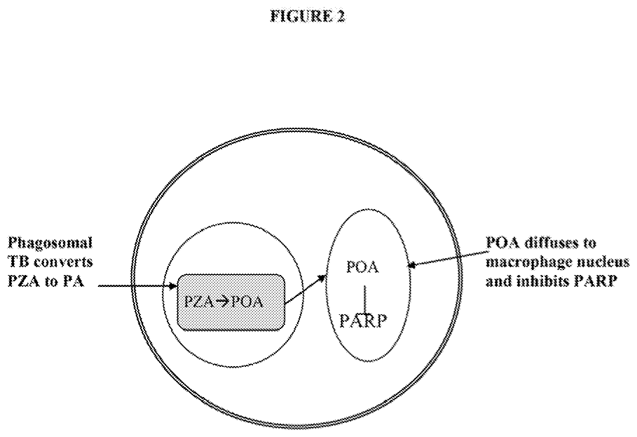

Furthermore, as is shown in FIG. 2, mycobacterially-derived POA will be formed within the phagosomes of TB infected macrophages, and so likely to achieve higher intracellular concentrations than in the plasma and thereby being even move dominant over PZA in inhibiting macrophage PARP. This is in accord with very recent data showing that systemically delivered POA is ineffective in vivo, despite achieving significant plasma levels.⁴⁵

Intersection of TB-Induced NAD⁺ Hydrolysis and PARP Inhibition upon Macrophage Necrosis

Although not the inventors' own work, it was recently shown that the mycobacterially-induced necrosis in RAW 264.7 macrophages was strongly inhibited by PARP inhibition (by 3-aminobenzamide, 3-AB).¹¹ Furthermore, it was also recently shown that the tuberculosis necrotizing toxin (TNT)⁴⁶ acts to cause necrosis in RAW 264.7 macrophages by hydrolyzing macrophage NAD⁺.⁴⁷ Since PARP activation causes significant NAD⁺ depletion that mediates cell death,⁴⁸ the inventors postulate that PARP inhibition by POA will counteract NAD⁺ depletion by TNT in macrophages, preventing macrophage necrosis and thereby preventing loss of this important antimycobacterial cell type.

Determine the Role of PARP inhibition by POA and PZA in the mycobacteria-macrophage interface. Using biochemical and cell biological approaches will examine the consequences of PARP inhibition in enzyme and cell based systems.

Rationale Although the inventors demonstrate PARP inhibition at relevant concentrations of POA and perhaps PZA, and other data shows the importance of maintaining macrophage NAD⁺ levels, there remains a profound need both to demonstrate the importance of POA in preventing macrophage necrosis and to elucidate the molecular mechanisms involved. Doing so will test our hypothesis that POA inhibition of macrophage PARP contributes to the antimycobacterial activity of pyrazinamide by preventing NAD⁺ depletion and necrosis. In vitro enzymology of PARP inhibition by POA and PZA.

Experimental Approach Although the inventors have demonstrated the whole-cell efficacy essential for meaningful antimycobacterial activity, the IC₅₀ from these experiments could be confounded by differential uptake and efflux of PZA and/or POA. Firstly, the inventors will immune-precipitate PARP from RAW 264.7 macrophages as the inventors have previously in keratinocyte cell lines.^{2,40} The RAW 264.7 macrophages will have received a) no PARP stimulation, b) PARP stimulation by UV, and c) PARP stimulation by co-infection with M. bovis BCG at a multiplicity of infection of infection (MOI) of 10 as previously.¹¹ The inventors will determine the IC₅₀ for PARP inhibition in these immunoprecipitates as previously using the HT Colorimetric PARP/Apoptosis Assay kit (Trevigen, Gaithersburg, Md.) according to the manufacturer's instructions (as in FIG. 1). This approach is also used to study the potential antagonism of POA PZA inhibition of PARP by INH and INH metabolites.

Demonstration that POA PZA are Able to Prevent Mycobacterial Macrophage NAD⁺ Depletion and Necrosis.

Experimental Approach The inventors first use RAW 264.7 cell lines, but later use primary murine bone marrow-derived macrophages (BMM) that are cultured from femurs of C57B1/6 WT mice and maintained in media containing DMEM, 4 mM L-glutamine, 20% FBS, 30% Macrophage-colony stimulating factor, as previously in our group.⁵⁰⁻⁵² Macrophage cells and cell lines will be infected with M. bovis BCG in BSL2 conditions at an MOI of 10, and incubated with varying concentrations of either POA or PZA (0 to 500 μM), for times between 2 and 48 hours. Then cells will be harvested and either fixed (4% paraformaldehyde) for microscopy/flow cytometry, or lysed and filtered through 0.2 micron filters for biochemical assays. M. bovis BCG is a natural mutant in pyrazinamidase,⁵³ and so will not convert PZA into POA, allowing their study in isolation. The inventors use flow cytometry to determine necrosis as reported,¹¹ immunohistochemistry and/or flow cytometry to study the overall poly-ADP ribose production, the inventors will determine cellular NAD⁺ levels by fluorescence using the EnzyFluo NAD/NADH kit (BioAssay Sytems, Hayward Calif.),⁴⁷ and the inventors determine PARP activity as previously. Again, these assays will be used to study antagonism,

### The Role of Mycobacterial POA Production in Macrophage Necrosis

Experimental Approach Because of differences in virulence on M. bovis BCG and pyrazinamidase positive M. tuberculosis H37Rv, these cannot be used to compare the effect of PZA conversion to POA in macrophage studies. Therefore, isogenic wild-type and PZA resistant mutants in M. tuberculosis H37Rv will be used, the latter developed by treatment with sub-MIC levels of PZA, and their loss of pyrazinamidase confirmed by negative Wayne test.⁵⁴ The inventors will use these strains to infect RAW 264.7 and BMM cells as in above in objective 1.2, but at BSL-3, and analyze for necrosis. PARP activity and NAD⁺ levels as above. While the inventors expect POA to equally inhibit necrosis, NAD⁺ loss and inhibit PARP for both wild-type and PZAse deficient M. tuberculosis. The inventors predict that PZAse deficient mycobacteria will not benefit as much from PZA as POA, due to a lack of POA production. This will definitively test our hypothesis (shown in FIG. 2) that mycobacterially-derived POA can strongly affect the macrophage PARP-NAD⁺ system.

### Genetic and Molecular Approaches to Recapitulate Pharmacology Findings

Experimental Approach Although the pharmacological approaches in objectives 1.1-1.3 are most appropriate to examine a new activity of an old drug. The inventors must be alert of the possibility for polypharmacology and off-target effects also being relevant. We will use PARP 1 & 2 knockdowns,⁵⁵ to confirm that PARP depletion of NAD⁺ drives induction of necrosis and that PARP inhibition is a meaningful mechanisms of action of POA/PZA. PARP-1 knockout mice are available from Jackson, and could be used to provide BMMs if necessary. Currently, CpnT mutants are not commercially available in M. tuberculosis, but are described in both M. tuberculosis and M. bovis BCG from NIH funded work, so we anticipate availability of these mutants also.⁴⁶ We will use these mutants in experiments as in objective 1.2, anticipating that less NAD⁺ loss will drive lower necrosis, and require lower amounts of POA or PZA to overcome either.

### Results, Possible Pitfalls, Alternative Approaches, and Experimental Robustness Reproducibility and Quality Control

The inventors expect that isolated enzyme IC₅₀ values in objective 1.1 mirror those from cells as although POA efflux from mycobacteria is known,⁵⁶ it is not in mammalian cells. Should great differences occur. The inventors will measure intracellular and free PZA and POA by HPLC, to determine if macrophage efflux is responsible. In objective 1.2, the inventors anticipate that POA and PZA have beneficial effects upon macrophage necrosis and NAD⁺ levels, at concentrations that mirror their abilities to inhibit PARP. To confirm that NAD⁺ hydrolysis and depletion drive cell outcomes, the inventors conduct key experimental conditions (low to zero POA/PZA), but supplement cells with 5 mM nicotinamide and 10 μM nicotinic acid, that are known to rescue NAD⁺ levels.⁴⁷ In objective 1.3, the inventors expect POA to equally inhibit necrosis, NAD⁺ loss and inhibit PARP for both wild-type and PZAse deficient M. tuberculosis. However, the inventors predict that PZAse deficient mycobacteria do not benefit as much from PZA as POA, due to lack of POA production. In this objective, the inventors also use M. bovis BCG with POA and PZA as an additional alternative approach, while remaining cognizant of its altered virulence.

The inventors obtain fresh vials of RAW cells and M. tuberculosis H37Rv from ATCC, so that the biological provenance of the cells used is known. Although the RAW 264.7 cell line is widely used, it remains a cell line, and so key findings will be replicated in primary BMM to ensure that they are relevant to non-immortalized cell lineages. In PARP inhibition studies, the inventors always use a range of positive PARP inhibitors (e.g. 3-AB or rucaparib⁵⁷) to ensure that a tack of observed inhibition is real and not a function of assay failure. Kits generally contain standards and are subject to manufacturer quality control, but the inventors investigate failures to operate in an expected manner. Should any chemical reagent not perform as expected, the inventors have it analyzed by mass spectrometry and proton NMR in the UNM Chemistry department, and TLC/HPLC as appropriate. Should there remain concerns, the inventors either order from an alternate supplier or synthesize in house. PZA and POA agents at differing dilutions are provided to the experimentalist in a blinded manner (i.e. simply randomly numbered samples) who will report data at an open lab meeting when the ‘coding’ is revealed to all incur labs: this prevents investigator bias for predetermined hypothesis-validating data. Where indicated, key experiments are re-performed by the Co-PI Zhou. Once key doses of POA and PZA and determined, the inventors will use these for completely independent experiments of reproducibility, and should less than 80% of successfully-replicated experiments show similar effects, the inventors are to further examine findings.

Antagonism of PARP inhibition by Isoniazid and its Metabolites

Because PARP is the first host target for PZA to be discovered, and because of structural similarities, antagonism of POA/PZA PARP inhibition is a logical possibility to explain the antagonism of PZA and INH that only occurs in the host.¹²⁻¹⁴ Furthermore, INH derived ROS/RNS might activate PARP. Biochemical and cellular approaches explore potential antagonistic interactions, both of INH and metabolites upon PARP inhibition and also by PARP activation by INH-derived reactive species.

Rationale It has long been known that PZA is antagonized by INH, only in vivo in a host,¹²⁻¹⁴ although the mechanism(s) of this antagonism have remained unknown. The inventors hypothesize that INH antagonizes PARP inhibition by PZA POA and thus result in this antagonism through two potential mechanisms a) direct competition at PARP between POA PZA and INH or its metabolites based upon similar molecular structure and or b) activation of PARP due to the wide range of DNA damaging reactive oxygen and nitrogen species generated by INH activation by KatG. Although speculative, the inventors believe the R21 mechanism appropriate for such a study, especially considering the strong evidence to support Aim 1, and the development of approaches in this aim.

### Investigate Direct Antagonism of PARP Inhibition by INH and Metabolites.

Experimental Approach The inventors use the in vitro PARP activity assay (described in objective 1.1) to examine whether INH or its metabolites can antagonize POA or PZA inhibition of PARP. The inventors use INH, isonicotinic acid and also the INH-NAD adduct, important in antitubercular activity, that are made by biomimetic Mn(III) pyrophosphate oxidation.^{58,59} Antagonism is detected by a shift in the IC₅₀ for PARP inhibition.

### Investigate PARP Activation by INH-derived ROS and RNS

Experimental Approach INH is activated to a range of ROS and RNS by mycobacterial KatG,^{15,17} and INH derived species are known to cause DNA damage,²⁵ and even activate PARP (in hepatocytes expressing P450, and at high dose, and likely not relevant to macrophages).⁶⁰ The inventors firstly use the macrophage cell line infected with M. tuberculosis as in objective 1.2, and examine PARP activation upon treatment of the infected macrophages with a range of INH concentrations, with KatG deletion and S315T mutants (that result in none and diminished NH activation) as controls. The inventors then determine the IC₅₀ for POA in whole cell PARP inhibition as previously, using an optimal INH concentration to determine if this PARP activation alters POA action. Finally, the inventors examine if INH enhances mycobacteria-induced macrophage necrosis as in objective 1.3, and determine if INH antagonized the protection afforded by POA or PZA.

Expected Results, Possible Pitfalls, Alternative Approaches, and Experimental. Robustness, Reproducibility and Quality Control

The same robustness, reproducibility and QC approaches detailed above is used. Should INH, INA or INH-NAD adduct show antagonism in vitro the inventors will also examine other known metabolites. The inventors expect to observe enhanced PARP activation in infected macrophages upon INH treatment, as many of the reactive species formed (e.g. NO, H₂O₂, OONO⁻and peroxides) have relatively long lifetimes and so are expected to diffuse into their nuclei and damage DNA. Should the inventors not see PARP activation, it may result from a lack of DNA damage, so the inventors will probe cellular DNA oxidation by immunoperoxidase staining for 8-OHdG as done previously.² The inventors are agnostic as to whether INH or metabolites will directly affects PARP inhibition by POA/PZA, should the inventors observe such inhibition they will examine INH or metabolite binding to PARP by Maldi-MS and absorption and fluorescence spectroscopy as previously,^{2,49} to determine binding affinity. One intriguing possibility is that INH derived ROS may act to oxidize PARP zinc-finger cysteine residues, as the inventors have observed for arsenic,⁵⁷ however, the inventors feel this is unlikely as it involved direct chelation of the zinc finger site by arsenic, that is not possible for INH, but should PARP activity decline with INH co-treatment we will study this as previously.⁶¹

### REFERENCES

1 Mitchison, D. A. Role of individual drugs in the chemotherapy of tuberculosis. Int J Tuberc Lung D 4, 796-806 (2000).
2 Sun, X. et al. Arsenite binding-induced zinc loss from PARP-1 is equivalent to zinc deficiency in reducing PARP-1 activity, leading to inhibition of DNA repair. Toxicology and applied pharmacology 274, 313-318 (2014).
3 Zhang, Y. et al. ‘Zs-MDR-TB’ versus ‘Zr-MDR-TB’: improving treatment of MDR-TB by identifying pyrazinamide susceptibility. Emerging Microbes & Infections 1, e5 (2012).
4 Chang. K.-C. et al. Pyrazinamide may improve fluoroquinolone-based treatment of multidrug-resistant tuberculosis. Antimicrobial Agents and Chemotherapy 56, 5465-5475 (2012).
5 Zhang, Y. & Mitchison, D. The curious characteristics of pyrazinamide: a review. The international journal of tuberculosis and lung disease: the official journal of the International Union against Tuberculosis and Lung Disease 7, 6-21 (2003).
6 Konno. K., Feldmann, F. M. & Mcdermot. W. Pyrazinamide Susceptibility and Amidase Activity of Tubercle Bacilli. American Review of Respiratory Disease 95. 461-(1967).
7 Zhang, Y. & Mitchison, D. The curious characteristics of pyrazinamide: a review, Int J Tuberc Lung D 7, 6-21 (2003).
8 Shi. W. et al. Pyrazinamide inhibits trans-translation in Mycobacterium tuberculosis. Science 333. 1630-1632, doi:10.1126/science. 1208813 (2011).
9 Shi. W. et al. Aspartate decarboxylase (PanD) as a new target of pyrazinamide in Mycobacterium tuberculosis. Emerg Microbes Infec 3, e58 (2014).
10 Via, L. et al. Host-mediated bioaciivation of pyrazinamide: Implications for efficacy, resistance, and therapeutic alternatives. ACS infectious diseases 1.203-214 (2015).
11 Wu. X. et al. Wnt/β-Catenin signaling reduces Bacillus Calmette-Guerin-induced macrophage necrosis through a ROS-mediated PARP/AIF-dependent pathway. BMC immunology 16, 1 (2015).
12 Grosset. J., TrufTot-Peinot. C, Lacroix. C. & Ji, B Antagonism between isoniazid and the combination pyrazinamide-rifampin against tuberculosis infection in mice. Antimicrobial Agents and Chemotherapy 36, 548-551 (1992).
13 Almeida, D. et al. Paradoxical effect of isoniazid on the activity of rifampin-pyrazinamide combination in a mouse model of tuberculosis. Antimicrobial Agents and Chemotherapy 53, 4178-4184 (2009).
14 Grosset. J. et al. Modeling early bactericidal activity in murine tuberculosis provides insights into the activity of isoniazid and pyrazinamide. Proceedings of the National Academy of Sciences 109, 15001-15005 (2012).
15 Timmins. G. S. & Deretic, V. Mechanisms of action of isoniazid. Mol Microbial 62, 1220-1227. doi:10.1111/j.1365-2958.2006.05467.x (2006).
16 Timmins. G. S., Master, S., Rusnak. F. & Deretic. V. Requirements for nitric oxide generation from isoniazid activation in vitro and inhibition of mycobacterial respiration in vivo. J Bacterial 186, 5427-5431 (2004).
17 Timmins, G. S., Master. S., Rusnak, F. & Deretic. V. Nitric oxide generated from isoniazid activation by KatG: source of nitric oxide and activity against Mycobacterium tuberculosis. Antimicrob Agents Chemother 48, 3006-3009 (2004).
18 Malone. L. et al. The Effect of Pyrazinamide (Aldinamide) on Experimental Tuberculosis in Mice. American Review of Tuberculosis and Pulmonary Diseases 65, 511-518 (1952).
19 Sololorovskv, M. et al. Pyrazinoic acid amide-An agent active against experimental murine tuberculosis. Experimental Biology and Medicine 79, 563-565 (1952).
20 Zhang, Y., Scorpio, A. Nikaido. H. & Sun. Z. Role of acid pH and deficient efflux of pyrazinoic acid in unique susceptibility of Mycobacterium tuberculosis to pyrazinamide. Journal of Bacteriolog 181, 2044-2049 (1999).
21 Wade, M. M. & Zhang. Y. Effects of weak acids, UV and proton motive force inhibitors on pyrazinamide activity against Mycobacterium tuberculosis in vitro. J Antimicrob Chemoth 58, 936-941 (2006)
22 Peterson, N. D., Rosen, B. C. Dillon. N. A. & Baughn, A. D. Uncoupling environmental pH and intrabacterial acidification from pyrazinamide susceptibility in Mycobacterium tuberculosis. Antimicrobial Agents and Chemotherapy 59, 7320-7326 (2015).
23 Mahon, R. N. & Hafner, R. Immune Cell Regulatory Pathways Unexplored as Host-Directed Therapeutic Targets for Mycobacterium tuberculosis: An Opportunity to Apply Precision Medicine Innovations to Infectious Diseases. Clinical infections diseases 61, S200-S216 (2015).
24 Almeida. D. V et al. Revisiting anti-tuberculosis activity of pyrazinamide in mice. Mycobacterial diseases: tuberculosis & leprosy 4, 145 (2014).
25 Ito. K , Yamamoto. K. & Kawanishi, S. Manganese-Mediated Oxidative Damage Of Cellular And Isolated Dna By Isoniazid And Related Hydrazines—Non-Fenton-Type Hydroxyl Radical Formation, Biochemistry 31, 11606-11613 (1992).
26 Schreiber. V., Dantzer. F., Arne. J.-C. & De Murcia. G. Poly (ADP-ribose): novel functions for an old molecule. Nature Reviews Molecular Cell Biology 7, 517-528 (2006).
27 Yee, D. P., Menzies, D. & Brassard, P. Clinical outcomes of pyrazinamide-monoresistant Mycobacterium tuberculosis in Quebec, Int J Tuberc Lung D 16, 604-609, doi:DOI 10.5588/ijtld. 11.0376 (2012).
28 Whitfield, M. G. et al. A Global Perspective on Pyrazinamidc Resistance: Systematic Review and Meta-Analysis, Plos One 10, e01 33869 (2015).
29 Murai. J. et al. Trapping of PARP1 and PARP2 by clinical PARP inhibitors. Cancer research 72, 5588-5599 (2012).
30 Hopkins. T. A. et al. Mechanistic dissection of PARP1 trapping and the impact on in vivo tolerability and efficacy of PARP inhibitors. Molecular Cancer Research 13, 1465-1477 (2015).
31 Wiggans, A. J., Cass. G., Bryant, A., Lawrie, T. A. & Morrison. J. Poly (ADP-ribose) polymerase (PARP) inhibitors for the treatment of ovarian cancer. Cochrane Database Syst Rev 5 (2015).
32 Choi, S. W. et al. Rapid in vivo detection of isoniazid-sensitive Mycobacterium tuberculosis by breath test. Nature communications 5, 4989, doi:10.1038/ncomms5989 (2014).
33 Timmins, G. et al. (USPTO 20150104819, 2015).
34 Timmins. G. & Choi, S. W. (USPTO 14/426,543., 2013).
35 Timmins, G. & Deretic, V. P. (U.S. Pat. No. 8,921,569, 2013).
36 Timmins, G., Master, S., Deretic, V. P. & Bishai, W. (U.S. Pat. No. 8,921,569, 2014).
37 Timmins. G. S., Choi. S. W. & Silks, L. P. Rapid Phenotype Tests For Antitubercular Drug Sensitivity And Resistance PCT/US2015/016349. (2015).
38 Timmins, G. S. Detecting virulence and drug-resistance mycobacterial phenotypes in vivo. Trends Microbiol 23, 321-323, doi:10.1016/j.tim.2015.02.013 (2015).
39 Maiga, M. et al. In vitro and in vivo studies of a rapid and selective breath test for tuberculosis based upon mycobacterial CO dehydrogenase. mBio 5, e00990, doi:10.1128/mBio.00990-14 (2014).
40 Jassal, M. S. et al. 13[C]-urea breath test as a novel point-of-care biomarker for tuberculosis treatment and diagnosis. Plos One 5, e12451, doi:10.1371/joumal.pone.0012451 (2010).
41 Siddiqui. S. D. NCT01301144 Oral Urea Breath Testing for Diagnosis and Treatment Response in Pulmonary Tuberculosis, 2011).
42 Raissy, H. NCT01303068 Detection of Pseudomonas Aeruginosa in the Airways of Patients With CF. (2011).
43 Raissy. H., Heynekamp, T., Davies, L., Morales, E. & Harkins, M. S. in American Thoracic Society A2859 (San Diego, 2014).
44 Prideaux. B. et al. The association between sterilizing activity and drug distribution into tuberculosis lesions. Nature Medicine 21,1223-1227 (2015).
45 Lanoix, J. P. et al. High systemic exposure of pyrazinoic acid has limited anti-tuberculosis activity in murine and rabbit models of tuberculosis. Antimicrob Agents Chemother Online Early, doi:10.1128/AAC.03085-15 (2016).
46 Danilchanka, O. et al. An outer membrane channel protein of Mycobacterium tuberculosis with exotoxin activity. Proceedings of the National Academy of Sciences III, 6750-6755 (2014).
47 Sim. J. et al. The tuberculosis necrotizing toxin kills macrophages by hydrolyzing NAD. Nature structural & molecular biology 22, 672-678 (2015).
48 Alano. C. C. et al. NAD+ Depletion Is Necessary and Sufficient for Poly (ADP-Ribose) Polymerase-1-Mediated Neuronal Death. The Journal of Neuroscience 30, 2967-2978 (2010).
49 Zhou, X. et al. Arsemte interacts selectively with zinc finger proteins containing C3H1 or C4 motifs. Journal of Biological Chemistry 286, 22855-22863 (2011).
50 Master, S. S. et al. Mycobacterium tuberculosis prevents inflammasome activation. Cell host & microbe 3, 224-232 (2008)
51 Miller, B. H. et al. Mycobacteria inhibit nitric oxide synthase recruitment to phagosomes during macrophage infection. Infection and Immunity 72, 2872-2878 (2004).
52 Castillo, E. F. et al. Autophagy protects against active tuberculosis by suppressing bacterial burden and inflammation. Proceedings of the National Academy of Sciences of the United States of America 109, E3168 (2012).
53 Scorpio, A. et al. Rapid differentiation of bovine and human tubercle bacilli based on a characteristic mutation in the bovine pyrazinamidase gene. Journal of clinical microbiology 35, 106-110 (1997).
54 Wayne, L. G. Simple pyrazinamidase and PARP tests for routine identification of mycobacteria. American Review of Respiratory Disease 109, 147-151 (1974).
55 Qin. X.-J. et al. Dual actions involved in arsenite-induced oxidative DMA damage. Chemical Research in Toxicology 21, 1806-1813 (2008).
56 Zimic, M. et al. Pyrazinoic acid efflux rate in Mycobacterium tuberculosis is a better proxy of pyrazinamide resistance. Tuberculosis 92, 84-91 (2012).
57 Wang, F. et al. Arsenite-induced ROS/RNS generation causes zinc loss and inhibits the activity of poly (ADP-ribose) polymerase-1. Free Radical Biology And Medicine 61, 249-256 (2013).
58 Nguyen, M., Claparols, C., Bemadou, J. & Meunier, B. A fast and efficient metal-medialed oxidation of isoniazid and identification of isoniaztd-NAD(H) adducts. Chem Bio chem 2, 877-883 (2001).
59 Nguyen, M., Quemard, A., Broussy. S., Bemadou. J, & Meunier. B. Mn(III) Pyrophosphate as an Efficient Tool for Studying the Mode of Action of isoniazid on the InhA Protein of Mycobacterium tuberculosis. Antimicrob Agents Chemother 46, 2137-2114. (2002).
60 Zhang. T.-G. et al. Induction of protective autophagy against apopiosis in HepG2 cells by isoniazid independent of the p38 signaling pathway. Toxicology Research 5, 963-972 (2016).
61 Zhou. X., Cooper. K. L., Sun, X., Liu, K. J. & Hudson. L. G. Selective Sensitization of Zinc Finger Protein Oxidation by Reactive Oxygen Species through Arsenic Binding. Journal of Biological Chemistry 290, 18361-18369 (2015).

1. A method of treating a Mycobacterium infection in a patient in need, comprising administering to said patient an effective amount of at least one PARP inhibitor.

2. The method according to claim 1 wherein said Mycobacterium infection is a Mycobacterium tuberculosis infection.

3. The method according to claim 1 wherein said PARP inhibitor is co-administcred in combination with at least one additional anti-tuberculosis agent.

4. The method according to claim 1 wherein said PARP inhibitor is selected from the group consisting of NU1025; 3-aminobenzamide; 4-amino-1,8-naphthalimide; 1,5-isoquinolinediol; 6(5H)-phenanthriddinone; 1,3,4,5,-tetrahydrobenzo(c)(1,6)- and (c)(1,7)-naphthyridin-6 ones; adenosine substituted 2,3-dihydro-1H-isoindol-1-ones; AG 14361; AG014699; 2-(4-chlorophenyl)-5-quinoxalinecarboxamide; 5-chloro-2-[3-(4-phenyl-3,6-dihydro-1 (2H)-pyridinyl)propyl]-4(3H)-quinazolinone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5 [4-(1-piperidinyl)(butoxy)-1(2H)-isoquinolone; CEP-6800; GB-15427; PJ34; DPQ; BS-201; BGB-290; BS401; CHP101; CHP102; E7016 (EISAI), INH2BP; BS1201; BS1401; TIQ-A; coumarin, benzamidc; picolinamidc, NMS-P118; E7449; NVP-TNKS656; G007-LK; ME0328; AZD2461; UPF 1069; an imidazobenzodiazepine PARP inhibitor; 8-hydroxy-2-methylquinazolinone (NU1025), CEP 9722, MK 4827, LT-673; 3-aminobenzamidc; ABT-888 (Veliparib); BSI-201 (Iniparib); Rucaparib (AG-014699); BMN-673 (Talazoparib); AZD2281 (Olaparib), INO-1001; A-966492; PJ-34; Niraparib (MK-4827), Arsenic trioxide (ATO), pharmaceutical salts and/or alternative salts thereof and mixtures thereof.

5. The method according to claim 1 wherein said PARP inhibitor is selected from the group consisting of ABT-888 (Veliparib), BSI-201 (Iniparib), Rucaparib (AG-014699), BMN-673 (Talazoparib), AZD2281 (Olaparib), Niraparib (MK-4827) and mixtures thereof.

6. The method according to claim 3 wherein said anti-tuberculosis agent is selected from the group consisting of isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin, pharmaceutical salts and/or alternative salts and mixtures thereof.

7. The method according to claim 3 wherein said anti-tuberculosis agent is or includes pyrazinamide and/or pyrazinoic acid.

8. The method according to claim 5 wherein said PARP inhibitor is Veliparib.

9. The method according to claim 2 wherein said Mycobacterium infection is a Mycobacerium tuberculosis infection and said infection is a recurrent, drug resistant and/or multiple drug resistant form of tuberculosis.

10. The method according to claim 9 wherein said infection is a drug resistant or multiple drug resistant form of tuberculosis.

11. The method according to claim 10 wherein said drug resistant form of tuberculosis is a PZA resistant form of tuberculosis.

12. A pharmaceutical composition comprising a combination of an amount of a PARP inhibitor with an additional anti-tuberculosis agent both included in said composition in amounts effective to treat a mycobacterium infection in a human patient in combination with a pharmaceutically acceptable carrier, additive or excipient.

13. The composition according to claim 12 in oral, parenteral or inhalation dosage form.

14. The composition according to claim 12 wherein said PARP inhibitor is selected from the group consisting of NU1025; 3-aminobenzamide; 4-amino-1,8-naphthalimide; 1,5-isoquinolinediol; 6(5H)-phenanthriddinone; 1,3,4,5,-tetrahydrobenzo (c)(1,6)-and (c)(1,7)-naphthyridin-6 ones; adenosine substituted 2,3 -dihydro-1H-isoindol-1-ones; AG14361; AG014699; 2-(4-chlorophenyl)-5-quinoxalinecarboxamide; 5-chloro-2-[3-(4-phenyl-3,6-dihydro-1(2H)-pyridinyl)propyl]-4(3H)-quinazolinone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5[4-(1-piperidinyl)(butoxy)-1(2H)-isoquinolone; CEP-6800; GB-15427; PJ34; DPQ; BS-201; BGB-290; BS401; CHP101; CHP102; E7016 (EISAI), INH2BP; BSI201; BSI401; TIQ-A; coumarin, benzamide; picolinamide, NMS-P118; E7449; NVP-TNKS656; G007-LK; ME0328; AZD2461; UPF 1069; an imidazobenzodiazepine PARP inhibitor; 8-hydroxy-2-methylquinazolinone (NU1025), CEP 9722, MK 4827, LT-673; 3-aminobenzamide; ABT-888 (Veliparib); BSI-201 (Iniparib); Rucaparib (AG-014699); BMN-673 (Talazoparib); AZD2281 (Olaparib), INO-1001; A-966492; PJ-34; Niraparib (MK-4827), Arsenic trioxide (ATO), pharmaceutical salts thereof and mixtures thereof.

15. The composition according to claim 12 wherein said PARP inhibitor is selected from the group consisting of ABT-888 (Veliparib), BSI-201 (Iniparib), Rucaparib (AG-014699), BMN-673 (Talazoparib), AZD2281 (Olaparib), Niraparib (MK-4827) and mixtures thereof.

16. The composition according to claim 12 wherein said anti-tuberculosis agent is selected from the group consisting of isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin, pharmaceutically acceptable salts, mixtures thereof.

17. The composition according to claim 12 wherein said anti-tuberculosis agent is or includes pyrazinamide and/or pyrazinoic acid.

18. The composition according to claim 12 wherein said PARP inhibitor is Veliparib.

19. The composition according to claim 12 in inhalation dosage form.

20. The composition according to claim 12 in oral dosage form.

21. The composition according to claim 12 in parenteral dosage form.

22. An assay for determining the activity of a compound as a potential anti-tuberculosis agent comprising poly ADP ribose polymerase (PARP) in combination with a reporter which can measure the inhibition on PARP of the unknown compound as evidenced by the reporter.

23. The assay according to claim 16 which is an ELISA assay.

24. A method for determining the activity of a compound as a potential anti-tuberculosis agent comprising exposing PARP in the assay according to claims 16 to a compound with unknown activity to be screened, obtaining a response of the unknown compound with PARP as evidenced by a response of the reporter in said assay and comparing said response of the reporter with a predetermined measurement wherein a measurement of said compound in said assay which is the same as, above or below the response of the reporter is an indication that the compound is an inhibitor of PARP and a potential anti-tuberculosis agent.

25.-32. (canceled)

33. A method for determining the activity of a compound as a potential anti-tuberculosis agent comprising exposing PARP in the assay according to claim 17 to a compound with unknown activity to be screened, obtaining a response of the unknown compound with PARP as evidenced by a response of the reporter in said assay and comparing said response of the reporter with a predetermined measurement wherein a measurement of said compound in said assay which is the same as, above or below the response of the reporter is an indication that the compound is an inhibitor of PARP and a potential anti-tuberculosis agent.
